(19) United States
(12) Patent Application Publication
(10) Pub. No.: US 2012/0290021 A1
(43) Pub. Date: Nov. 15, 2012

(54) BATTERY FEEDTHROUGH FOR AN IMPLANTABLE MEDICAL DEVICE

(75) Inventors: Sandeep Saurkar, St. Louis Park, MN (US); Gregory P. Shipe, Plymouth, MN (US); William J. Taylor, Anoka, MN (US); Jeffrey S. Lund, Forest Lake, MN (US); Bernard F. Heller, JR., Shoreview, MN (US)

(73) Assignee: Medtronic, Inc., Minneapolis, MN (US)

(21) Appl. No.: 13/104,715

(22) Filed: May 10, 2011

(51) Int. Cl. A61N 1/36; H01M 2/30; H01M 2/08

(52) U.S. Cl. 607/2; 429/181

(57) ABSTRACT

A battery feedthrough comprises a ferrule having a passage extending from a first side to a second side, a pin extending through the passage, an insulation sleeve disposed between the pin and the ferrule within the passage, there being a first junction between the pin and the insulation sleeve and a second junction between the insulation sleeve and the ferrule, and a coating comprising a polymer disposed over the pin on the first side of the ferrule, wherein the coating is formed by forming a preform comprising the polymer, placing the preform around at least a first portion of the pin on the first side of the ferrule, and melting the preform so that the polymer substantially covers a second portion of the pin, the first junction, a portion of the insulation sleeve, the second junction, and a portion of the ferrule on the first side of the ferrule.

### TECHNICAL FIELD

The present disclosure relates to a battery feedthrough, and in particular for a coating material for a battery feedthrough for an implantable medical device.

### BACKGROUND

Implantable medical devices typically rely on battery power to perform their therapeutic or diagnostic tasks. A battery supplies power to electrical components within the implantable medical device. A battery used with implantable medical devices may typically comprise chemical materials that provide for one or more electrochemical cells that produce electricity. These chemicals are often corrosive to the other materials within the implantable medical device. Therefore, the battery is typically configured with a battery feedthrough to permit a conductor to carry electrical current from the one or more electrochemical cells while keeping the corrosive materials contained within the battery.

### SUMMARY

In general, the present disclosure is directed to a coating that is formed on an interior portion of a battery feedthrough. The coating may help prevent the formation of potential electrical shorts at the feedthrough, such as shorts that may occur due to “lithium ball” formation, and also may prevent or reduce low leakage current between the conductor pin being passed through the feedthrough and a ferrule of the feedthrough. The coating material of the present disclosure is formed by forming a preform out of the coating material, placing the preform around the conductor pin, and melting the preform so that the coating material substantially covers a portion of the conductor pin, a portion of the ferrule, and a portion of an insulation sleeve that is disposed between the pin and the ferrule. In one example, the preform is made by extruding the coating material to form a small tubular preform.

In one example, the present disclosure is directed to a battery feedthrough comprising a ferrule having a first side, a second side, and a passage extending from the first side to the second side, a pin for conducting electricity between the first side of the ferrule and the second side of the ferrule, the pin extending through the passage, an insulation sleeve disposed between the pin and the ferrule within the passage, wherein the insulation sleeve electrically isolates the pin from the ferrule, there being a first junction between the pin and the insulation sleeve and a second junction between the insulation sleeve and the ferrule, and a coating comprising a polymer disposed over the pin on the first side of the ferrule, wherein the coating is formed by forming a preform comprising the polymer, placing the preform around at least a first portion of the pin on the first side of the ferrule, and melting the preform so that the polymer substantially covers a second portion of the pin, the first junction, a portion of the insulation sleeve, the second junction, and a portion of the ferrule on the first side of the ferrule.

In another example, the present disclosure is directed to an implantable medical device comprising a device housing, electronics located within the device housing, the electronics being configured to provide for a medical therapy, a battery located within the device housing, the battery comprising a battery housing enclosing an electrochemical battery cell, and a feedthrough comprising a ferrule mounted in an opening in the battery housing, the ferrule having an interior side disposed within the battery housing, an exterior side disposed outside the battery housing, and a passage extending between the interior side and the exterior side, a pin for conducting electrical current between the electrochemical battery cell and the electronics, the pin extending through the passage, an insulation sleeve disposed between the pin and the ferrule within the passage, wherein the insulation sleeve electrically isolates the pin from the ferrule, there being a first junction between the pin and the insulation sleeve and a second junction between the insulation sleeve and the ferrule, and a coating comprising a polymer disposed over the pin on the interior side of the ferrule, wherein the coating is formed by forming a preform from the polymer, placing the preform around at least a first portion of the pin on the first side of the ferrule, and melting the preform so that the polymer flows to cover a second portion of the pin, the first junction, a portion of the insulation sleeve, the second junction, and a portion of the ferrule on the interior side of the ferrule.

In another example, the present invention is directed to a method comprising forming a preform comprising a polymer, placing the preform around at least a first portion of a pin extending through a passage of a ferrule, wherein an insulation sleeve is disposed in the passage between the pin and the ferrule, there being a first junction between the pin and the insulation sleeve and a second junction between the insulation sleeve and the ferrule, and melting the preform so that the polymer flows to substantially cover a second portion of the pin, the first junction, a portion of the insulation sleeve, the second junction, and a portion of the ferrule.

This summary is intended to provide an overview of the subject matter described in this disclosure. It is not intended to provide an exclusive or exhaustive explanation of the techniques as described in detail within the accompanying drawings and description below. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example therapy system comprising an implantable medical device that may be used to monitor one or more physiological parameters of a patient and/or provide therapy to the patient.

FIG. 2 is a conceptual diagram illustrating another example therapy system comprising an implantable medical device that is implantable within the heart of the patient.

FIG. 3 illustrates the implantable medical device of FIG. 1 in further detail.

FIG. 4 is a partial cutaway view of an example implantable medical device.

FIGS. 5A-5C are cross-sectional views of an example battery feedthrough illustrating steps for coating portions of the battery feedthrough with a coating material.

FIGS. 6A-6C are perspective views of the example battery feedthrough of FIGS. 5A-5C illustrating the steps for coating portions of the battery feedthrough with the coating material.

FIG. 7 is a flow chart illustrating an example method of forming a battery feedthrough that may be used with an implantable medical device.

### DETAILED DESCRIPTION

In general, the present disclosure is directed to techniques for coating portions of a battery feedthrough usable with a medical device, such as an implantable medical device (IMD), such that the potential for internal short circuits between a conductor passing through the feedthrough and the feedthrough ferrule is reduced and so that low leakage current between the pin and feedthrough is reduced. The coating is formed by forming a preform from a polymer used to make the coating, placing the preform around the conductor pin, and melting the preform so that the polymer flows to substantially cover a portion of the pin, a portion of the ferrule, and a portion of an insulation sleeve disposed between the pin and the ferrule. In some examples, the preform is made by extruding the polymer to form a generally tubular-shaped preform that the pin is inserted through. The method of forming a preform, placing the preform around the pin, and melting the preform provides for precise control over the placement of the final coating and provides for more efficient manufacture of the battery feedthrough.

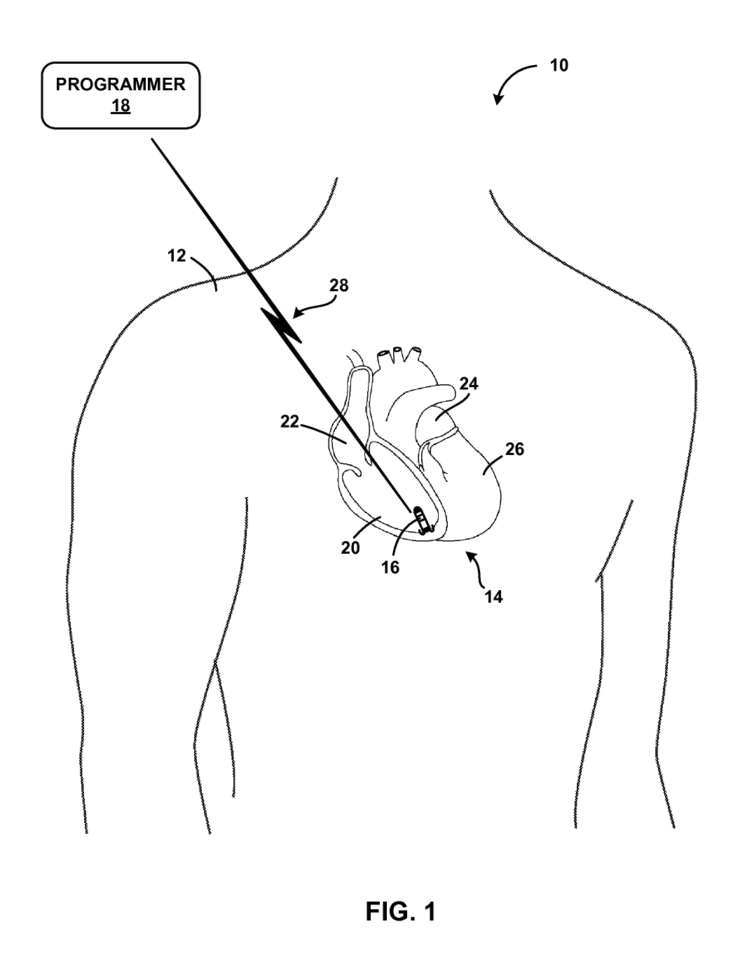

FIG. 1 is a conceptual diagram illustrating an example therapy system 10 that may be used to monitor one or more physiological parameters of a patient 12 and/or to provide therapy to the heart 14 of patient 12. Therapy system 10 includes IMD 16, which is coupled to a programmer 18. In the example shown in FIG. 1, IMD 16 is an implantable leadless pacemaker implantable within heart 14 of patient 12, wherein the leadless pacemaker may provide electrical signals to heart 14 via one or more electrodes on its outer housing (not shown in FIG. 1). In the example shown in FIG. 1, IMD 16 is sized to be implantable within a chamber of heart 14, such as right ventricle 20, as shown in FIG. 1, without substantially affecting heart 14 or substantially adversely affecting cardiac function. Additionally or alternatively, IMD 16 may sense electrical signals attendant to the depolarization and repolarization of heart 14 via electrodes on its outer housing. In some examples, IMD 16 provides pacing pulses to heart 14 based on the electrical signals sensed within heart 14.

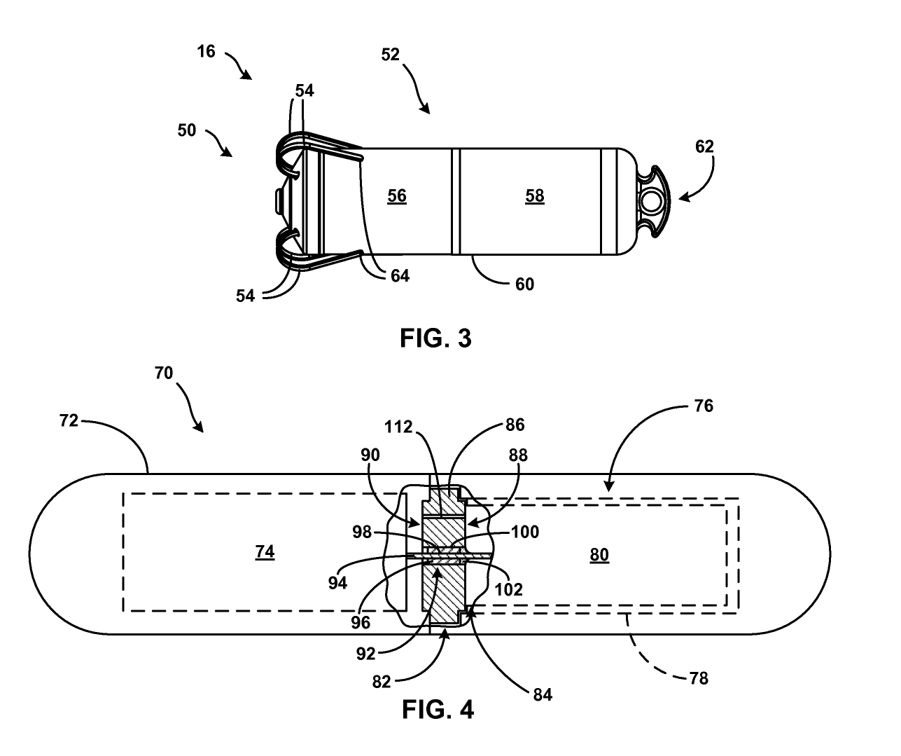

IMD 16 may include a fixation assembly, such as a set of active fixation tines, to secure IMD 16 to a patient tissue (described below with respect to FIG. 3). In other examples, IMD 16 may be secured with other techniques such as a helical screw or with an expandable fixation element. In the example of FIG. 1, IMD 16 is positioned wholly within heart 14 proximate to an inner wall of right ventricle 20 to provide right ventricular (RV) pacing. Although IMD 16 is shown within heart 14 and proximate to an inner wall of right ventricle 20 in the example of FIG. 1, IMD 16 may be positioned at any other location outside or within heart 14. For example, IMD 16 may be positioned outside or within right atrium 22, left atrium 24, and/or left ventricle 26, e.g., to provide right atrial, left atrial, and left ventricular pacing, respectively.

Depending on the location of implant, IMD 16 may include other stimulation functionalities. For example, IMD 16 may provide atrioventricular nodal stimulation, fat pad stimulation, vagal stimulation, or other types of neurostimulation. In other examples, IMD 16 may be a monitor that senses one or more parameters of heart 14 and may not provide any stimulation functionality. In some examples, system 16 may include a plurality of leadless IMDs 16, e.g., to provide stimulation and/or sensing at a variety of locations.

FIG. 1 further depicts programmer 18 in wireless communication with IMD 16 via a wireless communications link 28. In some examples, programmer 18 comprises a handheld computing device, computer workstation, or networked computing device. In one example, programmer 18 comprises a user interface that presents information to and receives input from a user, such as a physician, technician, surgeon, electrophysiologist, other clinician, or patient 12. It should be noted that the user may also interact with programmer 18 remotely via a networked computing device.

A user interacts with programmer 18 to communicate with IMD 16. For example, the user may interact with programmer 18 to retrieve physiological or diagnostic information from IMD 16. A user may also interact with programmer 18 to program IMD 16, e.g., select values for operational parameters of the IMD 16. For example, the user may use programmer 18 to retrieve information from IMD 16 regarding the rhythm of heart 14, trends therein over time, or arrhythmic episodes.

As an example, the user may use programmer 18 to retrieve information from IMD 16 regarding other sensed physiological parameters of patient 12 or information derived from sensed physiological parameters, such as intracardiac or intravascular pressure, intracardiac or intravascular fluid flow, activity, posture, tissue oxygen levels, respiration, tissue perfusion, heart sounds, cardiac electrogram (EGM), intracardiac impedance, or thoracic impedance. In some examples, the user may use programmer 18 to retrieve information from IMD 16 regarding the performance or integrity of IMD 16 or other components of system 16, or a power source of IMD 16. As another example, the user may interact with programmer 18 to program, e.g., select parameters for, therapies provided by IMD 16, such as pacing and, optionally, neurostimulation.

IMD 16 and programmer 18 may communicate via wireless communication link 28 using any techniques known in the art. Examples of communication techniques may include, for example, low frequency or radiofrequency (RF) telemetry, but other techniques are also contemplated. In some examples, programmer 18 may include a programming head that may be placed proximate to the patient's body near the IMD 16 implant site in order to improve the quality or security of communication between IMD 16 and programmer 18.

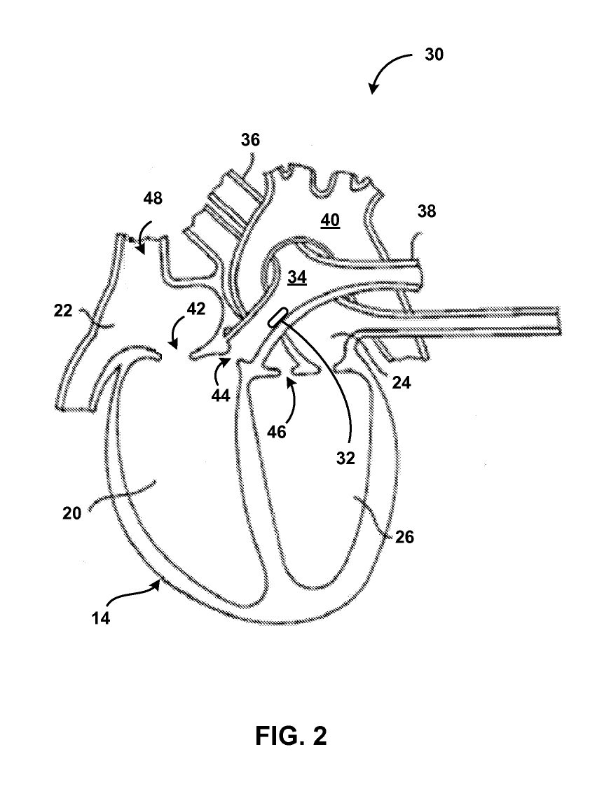

FIG. 2 is a conceptual diagram illustrating another example therapy system 30 comprising an IMD 32 implanted within another portion of heart 14, such as the pulmonary artery 34, as shown in FIG. 2. In one example, IMD 32 is a stimulation device, similar to the example of a leadless pacemaker described above with respect to IMD 16 of FIG. 1. In another example, IMD 32 is a sensing device, such as a pressure sensor 32 that provide for pressure measurements within patient 12, such as within the heart 14 of the patient. FIG. 2 is a diagram of a human heart 14 with pressure sensor 32 implanted therein. FIG. 2 depicts pulmonary artery 34, right atrium 22, right ventricle 20, left atrium 24, left ventricle 26, right pulmonary artery 36, left pulmonary artery 38, aorta 40, atrioventricular valve 42, pulmonary valve 44, aortic valve 46, and superior vena cava 48 of heart 14. Pressure sensor 32 may, as shown in FIG. 2, be placed inside pulmonary artery 34 of heart 14. In some example implementations, pressure sensor 32 may be placed within main pulmonary artery 34, the right pulmonary artery 36 or any of its branches, and/or within left pulmonary artery 38 or any of its branches, or within right ventricle 20. In other example implementations, multiple pressure sensors 32 may be placed at various locations within pulmonary artery 34, right pulmonary artery 36 or any of its branches, and/or left pulmonary artery 38 or any of its branches.

As shown in FIG. 2, pressure sensor 32 may be a leadless assembly, e.g., need not be coupled to another IMD or other device via a lead, and need not otherwise be coupled to any leads. Although not depicted, pressure sensor 32 may include wireless communication capabilities such as low frequency or radiofrequency (RF) telemetry, as well other wireless communication techniques that allow pressure sensor 32 to communicate with a separate IMD, such as IMD 16, a programming device, such as programmer 18, or another device. Pressure sensor 32 may be affixed to the wall of the pulmonary artery or the wall of the right ventricle using any number of fixation techniques. For example, pressure sensor 32 may include fixation elements, e.g., helical tines, hooked tines, barbs, or the like, that allow pressure sensor 32 to be secured to pulmonary artery 34. In other examples, pressure sensor 32 may be attached to a stent having any variety of conformations, for example, and the stent/sensor combination may be implanted within one or more of pulmonary artery 34, right pulmonary artery 36 or one of its branches, or left pulmonary artery 38 or one of its branches.

Pressure sensor 32 may be implanted within pulmonary artery 34, for example, using a delivery catheter. For example, a physician may deliver pressure sensor(s) 32 transvenously via a delivery catheter through either the internal jugular or femoral veins. The delivery catheter then extends through superior vena cava 48, right atrioventricular valve 42, right ventricle 20, and pulmonary valve 44 into pulmonary artery 34. In other examples, pressure sensor 32 may be implanted after a physician has opened the patient's chest by cutting through the sternum.

Pressure sensor 32 generates pressure information representing a pressure signal as a function of the fluid pressure in pulmonary artery 34, for example. Pressure sensor 32 may transmit the signal to another device, such as IMD 16, programmer 18, and/or another device, such as external monitoring equipment, which may receive, monitor, and analyze the pressure signal in order to determine a cardiac cycle length and/or other pressure metrics. In other examples, pressure sensor 32 may itself analyze the pressure information in order to determine a cardiac cycle length and/or other pressure metrics according to the techniques described herein. Further description of the collection and analysis of pressure data by pressure sensor 32 is described in U.S. Provisional Patent Application No. 61/368,437, titled “MEASUREMENT OF CARDIAC CYCLE LENGTH AND PRESSURE METRICS FROM PULMONARY ARTERIAL PRESSURE,” and filed Jul. 28, 2010, the entire contents of which are incorporated by reference as if reproduced herein.

FIG. 3 shows a close-up view of an example IMD 16 of FIG. 1. The example IMD 16 of FIG. 3 comprises a fixation subassembly 50 and an electronic subassembly 52. In one example, fixation subassembly 50 comprises active fixation tines 54 that are configured to be deployed in order to anchor IMD 16 to a patient tissue, such as a wall of heart 14. In one example, electronic subassembly 52 includes control electronics 56, which control the sensing and/or therapy functions of IMD 16, and battery 58, which powers control electronics 56. As one example, control electronics 56 may include sensing circuitry, a stimulation generator and a telemetry module. As one example, battery 58 may comprise features of the batteries disclosed in U.S. patent application Ser. No. 12/696,890, titled IMPLANTABLE MEDICAL DEVICE BATTERY and filed Jan. 29, 2010, the entire contents of which are incorporated by reference herein.

IMD 16 may also comprise a device housing 60 that encloses control electronics 56 and battery 58. Device housing 60 is formed from a biocompatible material, such as a stainless steel or titanium alloy. In some examples, the housings of control electronics 56 and battery 58 may include a parylene coating. IMD 16 may also include a delivery tool interface 62 that is configured to connect to a delivery device, such as a catheter, to deliver and position IMD 16 during implantation. In one example, shown in FIG. 3, delivery tool interface 62 is located at an end of device housing 60 proximal to electronic subassembly 52.

In one example, active fixation tines 54 are deployable from a spring-loaded position in which distal ends 64 of active fixation tines 54 point away from electronic subassembly 52 to a hooked position in which active fixation tines 54 bend back towards electronic subassembly 52. For example, active fixation tines 54 are shown in a hooked position in FIG. 3. Active fixation tines 54 may be fabricated of a shape memory material, which allows active fixation tines 54 to bend elastically from the hooked position to the spring-loaded position. As an example, the shape memory material may be shape memory alloy such as Nitinol.

In some examples, all or a portion of fixation subassembly 50, such as active fixation tines 54, may include one or more coatings. For example, fixation subassembly 50 may include a radio-opaque coating to provide visibility during fluoroscopy. In one such example, one or more of active fixation tines 54 may include one or more radio-opaque markers. As another example, active fixation tines 54 may be coated with a tissue growth promoter or a tissue growth inhibitor. A tissue growth promoter may be useful to increase the holding force of active fixation tines 54, whereas a tissue growth inhibitor may be useful to facilitate removal of IMD 16 during an explantation procedure, which may occur many years after the implantation of IMD 16.

As one example, IMD 16 and fixation subassembly 50 may comprise features of the fixation assemblies disclosed in U.S. Provisional Patent Application No. 61/428,067, titled, “IMPLANTABLE MEDICAL DEVICE FIXATION” and filed Dec. 29, 2010, the entire contents of which are incorporated by reference herein. Examples of other fixation structures are described in U.S. Provisional Patent Application No. 61/428,127, titled “IMPLANTABLE MEDICAL DEVICE FIXATION TESTING,” filed on Dec. 29, 2010, assigned to the assignee of the present disclosure, the entire contents of which are incorporated herein by reference as if reproduced herein.

FIG. 4 is a conceptual diagram showing an example IMD 70 that may be used for the treatment or diagnosis of a patient. IMD 70 of FIG. 4 may represent a stimulation device, such as the example leadless pacemaker IMD 16 described above with reference to FIG. 1, or a sensing device, such as pressure sensor 32 described above with respect to FIG. 2. The concepts of IMD 70 of FIG. 4 may be used in other types of implantable medical devices. For example, the described techniques can be readily applied to provide a battery feedthrough for an IMD that provides electrical stimulation to a tissue site of patient 12 proximate a muscle, organ or nerve, such as a tissue proximate a vagus nerve, spinal cord, brain, stomach, pelvic floor or the like. The described techniques may also be used to provide a feedthrough for implantable sensors, such as, but not limited to, a pressure sensor, an electrocardiogram sensor, a fluid flow sensor, a tissue oxygen sensor, an accelerometer, a glucose sensor, a potassium sensor, a thermometer and/or other sensors. Moreover, the techniques may be used to operate an IMD that provides other types of therapy, such as drug delivery or infusion therapies. As such, description of these techniques in the context of cardiac rhythm management therapy should not be limiting of the techniques as broadly described in this disclosure.

In the example shown in FIG. 4, IMD 70 comprises a device housing 72 enclosing electronics (shown conceptually as block 74 in FIG. 4) and a battery 76. Electronics 74 provide for the therapeutic and/or diagnostic functionality of IMD 70, such as circuitry configured to provide for electrical shock or stimulation therapy to a patient via one or more electrodes and/or circuitry configured to monitor aspects of a patient's condition, such as bioelectric signal detection and analysis or pressure sensing and analysis. Battery 76 comprises a battery housing 78 enclosing one or more electrochemical battery cells 80. The one or more electrochemical battery cells 80 comprising one or more electrolytes in contact with one or both electrodes of battery cell 80. For example, battery 76 may comprise a primary battery (non-rechargeable) with a common electrolyte within which both the cathode and anode are immersed. In one example, the electrolyte may comprise a lithium and fluorine containing electrolyte, such as lithium hexafluoroarsenate (LiAsF₆). In one example, the electrolyte comprises LiAsF₆ having a molar concentration of about 1.0 molar in a solution of propylene carbonate (C₄H₆O₃) and 1,2-dimethoxyethane, also referred to as glyme (CH₃O(CH₂)₂OCH₃), for example a 50:50 solution (by volume) of propylene carbonate and glyme. In one example, the cathode may comprise a composite of silver vanadium oxide (AgₓV_{y}O_{z}) and fluorinated carbon fibers, and the anode may comprise lithium metal.

A feedthrough 82 mounted in an opening 84 of battery housing 78 provides a means for passing electrical energy from electrochemical battery cell(s) 80 to electronics 74 in order to energize electronics 74 with the energy generated by electrochemical battery cells(s) 80. In one example, feedthrough 82 comprises a ferrule 86 having a first side 88 and a second side 90. As shown in FIG. 4, first side 88, or interior side 88, is disposed within battery housing 78 while second side 90, or exterior side 90, is disposed outside battery housing 78. A passage 92 extends through ferrule 86 between interior side 88 and exterior side 90. A pin 94 extends through passage 92, wherein pin 94 comprises an electrically conductive material for conducting electrical energy from electrochemical battery cell(s) 80 to electronics 74. An insulation sleeve 96 is disposed between pin 94 and ferrule 86, wherein insulation sleeve 96 electrically isolates pin 94 from ferrule 86. A first junction 98 exists between pin 94 and insulation sleeve 96 and a second junction 100 exists between insulation sleeve 96 and ferrule 86. In one example, insulation sleeve 96 provides a first hermetic seal at first junction 98 between pin 94 and insulation sleeve 96 and a second hermetic seal at second junction 100 between insulation sleeve 96 and ferrule 86.

As shown in FIG. 4, a coating 102 is disposed over at least a portion of pin 94 on interior side 88 of ferrule 86, wherein coating 102 comprises a polymer that is configured to substantially cover a portion of pin 94 on interior side 88 of ferrule 86, first junction 98 on interior side 88 of ferrule 86, a portion of insulation sleeve 96 on interior side 88 of ferrule 86, second junction 100 on interior side 88 of ferrule 86, and a portion of ferrule 86 on interior side 88 of ferrule 86. As described in more detail below, coating 102 is formed by forming a preform from the polymer of coating 102, placing the preform around at least a portion of pin 94 on interior side 88 of ferrule 86, and melting the preform so that the polymer flows to substantially cover the portion of pin 94, first junction 98, the portion of insulation sleeve 96, second junction 100, and the portion of ferrule 86 on interior side 88 of ferrule 86. The portion of pin 94 that preform 120 is placed around and the portion of pin 94 that coating 102 substantially covers may be the same portion of pin 94, or they may be different portions of pin 94, or there may be overlap between the portion of pin 94 that preform 120 is placed around and the portion of pin 94 that coating 102 substantially covers.

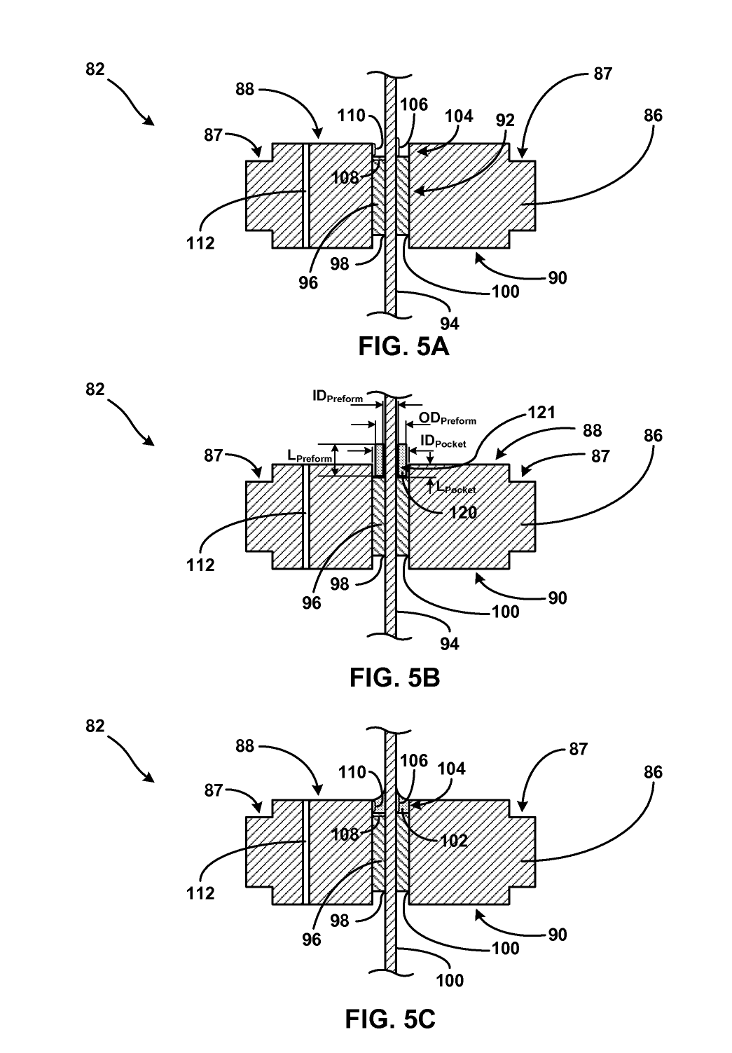

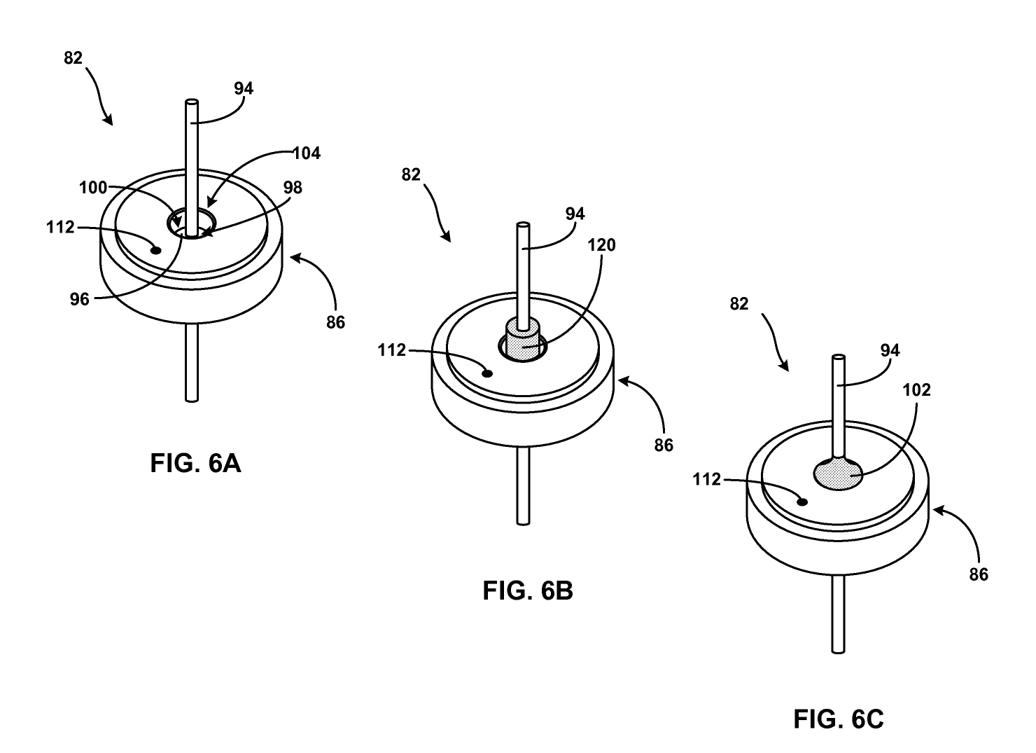

FIGS. 5A-5C and 6A-6C show feedthrough 82 of IMD 70 in greater detail. FIGS. 5A-5C show a cross-sectional view of feedthrough 82 through several example steps of forming coating 102 for feedthrough 82. FIGS. 6A-6C shows a perspective view of interior side 88 of ferrule 86 for the same respective example steps as in FIGS. 5A-5C. In one example, feedthrough 92 comprises a pocket 104 disposed around pin 94 on interior side 88 of ferrule 86. Pocket 104 may be formed as a depression or cavity within interior side 88 of ferrule 86 having a generally constant cross-sectional shape in the axial direction of ferrule 86. In one example, pocket 104 is defined by passage 92, which radially bounds pocket 104, and insulation sleeve 96, which bounds an axial end of pocket 104. In one example, best seen in FIG. 6A, pocket 104 is generally cylindrical, e.g. having a generally elliptical or circular cross-section, with a diameter that is larger than a diameter of pin 94. In one example, pocket 104 has a lateral width, such as the inner diameter of generally cylindrical pocket 104 shown in FIG. 6A, of between about 0.25 millimeters (about 0.0098 inches) and about 1 millimeter (about 0.0394 inches), such as between about 0.571 millimeters (0.0225 inches) and about 0.597 millimeters (about 0.0235 inches), for example about 0.584 millimeters (about 0.0230 inches).

Ferrule 86 may also comprise a weld zone 87 that provides a surface for welding ferrule 86 to housing 78 of battery 76 or device housing 72 of IMD 70. Weld zone 87 may comprise a profile that corresponds to a profile of the housing to which ferrule 86 is being welded. For example, as best seen in FIG. 5A, weld zone 87 may comprise a step or shoulder that corresponds to a profile of battery housing 78 (see FIG. 4).

In one example, ferrule 86 also comprises a fill port 112 that extends from interior side 88 to exterior side 90. Fill port 112 provides a pathway for filling electrochemical battery cell(s) 80 with electrolyte during manufacture of battery 76. In one example, fill port 112 is left open when making feedthrough 82 (e.g., when inserting pin 94 through passage 92, disposing insulation sleeve 96 between pin 94 and ferrule 86, forming coating 102 around pin 94), and when mounting the assembled feedthrough 82 in opening 84 in battery housing 82. The electrolyte material is then injected through fill port 112, e.g. with a needle or other injection apparatus, so that a desired amount of electrolyte material is within electrochemical battery cell(s) 80. After filling electrochemical battery cell(s) 80 with the electrolyte material, fill port 112 may be sealed, e.g., with a sealing weld, in order to prevent the electrolyte from leaking out through fill port 112.

Ferrule 86 may be made from any material that is practical for use with IMD 70. In one example, the material of ferrule 86 is generally easily formable into the desired shape of ferrule 86, e.g., through casting or machining ferrule 86, and is chemically inert to the electrolytes within battery 76. In one example, ferrule 86 comprises at least one of an aluminum-containing titanium alloy, such as Grade 23 titanium (e.g., between about 5.5 at. % and about 6.5 at. % aluminum (Al), between about 3.5 at. % and about 4.5 at. % vanadium (V), about 0.08 at. % carbon (C), about 0.13 at. % oxygen (O), and the balance titanium (Ti)), Grade 5 titanium, also known as Ti6Al4V titanium alloy (e.g., about 6 at. % Al, about 4 at. % V, less than about 0.08 at. % C, less than about 0.2 at. % O, less than about 0.05 at. % nitrogen (N), less than about 0.4 at. % iron (Fe), less than about 0.015 at. % hydrogen (H), and the balance Ti), or Grade 9 titanium, also known as Ti3Al2.5V (e.g., about 3 at. % Al, about 2.5 at. % V, less than about 0.05 at. % C, less than about 0.12 at. % O, less than about 0.02 at. % N, less than about 0.015 at. % H, and the balance Ti), a commercially pure titanium (e.g., Grade 1 titanium, Grade 2 titanium, Grade 3 titanium, and Grade 4 titanium), aluminum, or a stainless steel.

Pin 94 provides a conduction pathway for electrical energy generated by electrochemical battery cell 80 to electronics 74 of IMD 70. In one example, pin 94 is electrically coupled to an electrode within battery cell 80, such as by being welded to the anode or the cathode of battery cell 80. In one example, pin 94 is electrically connected to the cathode, and the anode is attached and electrically connected to an interior wall of battery housing 78. In another example, the electrical connections may be reversed, e.g. with a pin being electrically coupled to the anode and the cathode electrically coupled to an interior wall of the battery housing. The other end of pin 94 is electrically coupled to electronics 74, such as by being bonded or welded to a connection pad of electronics 74.

In one example, pin 94 comprises an electrically conductive material that is capable of carrying the desired current from battery 76 to electronics while still being substantially chemically inert to the electrolytes within battery cell 80. In one example, pin 94 comprises at least one of an aluminum-containing titanium alloy, such as Grade 23 titanium, Grade 5 titanium, or Grade 9 titanium, a niobium-containing titanium alloy, such as Grade 36 titanium (e.g., about 45 at. % niobium (Nb), and the balance titanium), a commercially pure titanium (e.g., Grade 1 titanium, Grade 2 titanium, Grade 3 titanium, and Grade 4 titanium), niobium, or stainless steel.

Pin 94 may have any cross-sectional shape that is practical for transmitting electrical energy from battery cell 80 to electronics 74. In one example, pin 94 has a cross-sectional shape that corresponds to the cross-sectional shape of pocket 104 on interior side 88 of ferrule 86. For example, as best seen in FIG. 6A, both pocket 104 and pin 94 are generally cylindrical with a generally circular cross-sectional shape, wherein the diameter of pin 94 is smaller than the diameter of pocket 104. In one example, pin 94 has a lateral width, such as the diameter of cylindrical pin 94 shown in FIG. 6A, of between about 0.05 millimeters (about 0.00197 inches) and about 0.635 millimeters (about 0.025 inches), such as between about 0.1 millimeters (about 0.0039 inches) and about 0.3 millimeters (about 0.0118 inches), for example about 0.203 millimeters (about 0.008 inches).

Insulation sleeve 96 electrically isolates pin 94 from ferrule 86 and also provides a seal to prevent the electrolyte material of battery cell 80 from leaking out of battery 76 through feedthrough 82. In one example, insulation sleeve 96 hermetically seals between pin 94 and ferrule 86, such as by providing a first hermetic seal between pin 94 and insulation sleeve (e.g., at first junction 98) and a second hermetic seal between ferrule 86 and insulation sleeve 96 (e.g., at second junction 100). In one example, insulation sleeve 96 comprises at least one of a boro-aluminate glass, such as LaBor-4 glass (e.g., glass having a molar concentration of about 30 B₂O₃, 20 CaO, about 20 Mg, about 15 Al₂O₃, about 10 SiO₂, and about 5 La₂O₃). Other glasses may be used to form insulation sleeve 96, such as CaBAl-12 glass (e.g., glass having a molar concentration of about 40 B₂O₃, about 20 Al₂O₃, about 20 MgO, and about 20 CaO), Ta-23 glass (e.g., glass having a weight % of about 45 wt. % SiO₂, about 20 wt. % Al₂O₃, about 12 wt. % CaO, about 8 wt. % B₂O₃, about 7 wt. % MgO, about 6 wt. % SrO, and about 2 wt. % La₂O₃), and Corning 9013 glass. Further description of potential materials of insulation sleeve are provided in the commonly-assigned U.S. Pat. No. 5,306,581 to Taylor et al, issued on Apr. 26, 1994, and U.S. Publication No. 2009/0321107 to Taylor et al., published on Dec. 31, 2009, the disclosures of which are incorporated by reference as if reproduced herein.

FIGS. 5A and 6A show feedthrough 82 after pin 94 has been inserted through ferrule 86 and after insulation sleeve 96 has been formed between pin 94 and ferrule 86, but before coating 102 has been formed around pin 94. As shown in FIG. 5A, within and/or proximate to pocket 104 is a small portion 106 of pin 94, a small portion 108 of insulation sleeve 96, and a small portion 110 of ferrule 86. As shown in the example of FIG. 5A, portion 106 of pin 94 and portion 108 of insulation sleeve 96 are on either side of first junction 98, while portion 108 of insulation sleeve 96 and portion 110 of ferrule 86 are on either side of second junction 100.

In some examples, feedthrough 82 as shown in FIGS. 5A and 6A (e.g., without coating 102) is capable of preventing electrolytes from leaking from battery cell 80 through passage 92. However, a feedthrough 92 without a coating, such as coating 102, may result in a low leakage current between pin 94 and ferrule 86. Another adverse effect involves the formation of structures between pin 94 and ferrule 86 from compounds within the electrolyte of battery cell 80. For example, if the electrolyte comprises a Li-containing compound, such as lithium hexafluoroarsenate (LiAsF₆), which may result in the formation of lithium metal structures, sometimes referred to as “lithium balls,” that bridge between pin 94 and ferrule 86. Because lithium is electrically conductive, the formation of lithium balls can result in electrical short circuits between pin 94 and ferrule 86.

It has been found that a coating material may be applied around pin 94 that reduces or prevents low leakage current between pin 94 and ferrule 86 and may also prevent the formation of lithium balls or other potential short circuiting structures between pin 94 and ferrule 86. The coating material is electrically insulating, and in some examples, prevents the formation of lithium balls or other short-circuiting structures on the surface of the coating.

One material that has been found to be useful for coating pin 94 is ethylene tetrafluoroethylene (ETFE). In some feedthroughs, a suspension of ETFE powder in a liquid (such as ethanol) is applied onto a pin, ferrule, or insulation sleeve desired to be coated with ETFE. The liquid may then evaporated off leaving behind the ETFE powder generally in the desired location for the coating. After evaporating the liquid from the ETFE powder, the ETFE powder may be melted so that the ETFE material flows around the pin and/or the ferrule and/or the insulation sleeve to form a coating. In some cases, multiple cycles of applying a coat of the ETFE suspension, evaporating the liquid, and melting the deposited powder may be performed so that there is adequate coverage and adequate thickness of the final coating.

The process of coating the pin and/or the ferrule and/or the insulation sleeve with the ETFE solution may be labor intensive and time consuming. Moreover, for very small feedthroughs, it may be difficult or impossible to apply the ETFE suspension only to the portion of the feedthrough upon which an ETFE coating is desired. For example, as described above, in some examples of feedthrough 82 shown in FIGS. 5A and 6A, pin 94 has an outer diameter of between about 0.1 millimeters (about 0.0039 inches) and about 0.3 millimeters (about 0.0118 inches) and pocket 104 of ferrule 86 has an inner diameter of between about 0.571 millimeters (about 0.0225 inches) and about 0.597 millimeters (about 0.0235 inches). At this scale, it may be difficult to apply the ETFE suspension only within or proximate pocket 104. Even if the suspension is successfully applied only within or proximate to pocket 104, after evaporation of the liquid, some of the ETFE powder may become dispersed away from pocket 104 and settle in other portions of feedthrough 82. For example, the ETFE powder is known to disperse to feed port 112 or the portions of ferrule 86 that are welded to battery housing 78 where the ETFE powder may detrimentally affect the weld intended to seal fill port 112 or the weld between ferrule 86 and battery housing 78. In some cases, the adversely-affected welds may result in leakage of the electrolyte material from battery 76.

In some examples, the present disclosure may provide a solution to the detrimental results that occurr when forming a coating by applying an ETFE-powder suspension. Rather than using a suspension of the coating material, such as ETFE, the present disclosure may use a preform 120 (FIGS. 5B and 6B) made from the coating material. In some examples, described in more detail below, preform 120 comprises a geometry that is selected to provide substantially complete coverage by coating 102 of portion 106 of pin 94, portion 108 of insulation sleeve 96, portion 110 of ferrule 86, and first junction 98 and second junction 100 on interior side 88 of ferrule 86. In some examples, the geometry of preform 120 is selected to provide a conformal coating 102 that substantially covers all of portion 106 of pin 94, first junction 98, portion 108 of insulation sleeve 96, second junction 100, and portion 110 of ferrule 86.

Preform 120 may provide for easy placement at a desired location of coating 102 because preform 120 may be an easily manipulatable, solid structure that can easily be placed around pin 94 prior to melting. Preform 120 may also allow the coating material to be placed only in the desired location so that some of the coating material will not be dispersed to undesired locations of the feedthrough or IMD. Further, as described with respect to the Examples below, preform 120 may provide for a reduction in manufacturing time and costs.

FIGS. 5B and 6B show an example preform 120 that has been formed and placed around at least a portion of pin 94. As noted above, preform 120 is configured so that when it is melted, preform 120 forms a coating 102 that substantially covers a portion of pin 94, such as portion 106, a portion of insulation sleeve 96, such as portion 108, and a portion of ferrule 86, such as portion 110, as well as first junction 98 between pin 94 and insulation sleeve 96 and second junction 100 between insulation sleeve 96 and ferrule 86. In one example, preform 120 is configured so that when it is melted, preform 120 forms a coating 102 that substantially fills pocket 104, such as by forming a conformal coating on portion 106 of pin 94, portion 108 of insulation sleeve 96, and portion 110 of ferrule 86.

In one example, preform 120 comprises a lumen 121 through which pin 94 is inserted. In the example shown in FIGS. 5B and 6B, wherein pin 94 and pocket 104 are each generally cylindrical, preform 120 is also generally cylindrical with an inner diameter ID_{Preform} that is larger than the diameter of pin 94 (not labeled) and an outer diameter OD_{Preform} that is smaller than the inner diameter ID_{Pocket} of pocket 104. In one example, preform 120 may have an inner diameter ID_{Preform} that is between about 0.05 millimeters (about 0.00197 inches) and about 0.55 millimeters (about 0.0217 inches), for example between about 0.1 millimeters (0.0039 inches) and about 0.35 millimeters (about 0.0138 inches), such as about 0.25 millimeters (about 0.00984 inches). In one example, preform 120 may have an outer diameter OD_{Preform} of between about 0.24 millimeters (about 0.00945 inches) and about 1 millimeter (about 0.0394 inches), for example between about 0.5 millimeters (about 0.0197 inches) and about 0.597 millimeters (about 0.0235 inches), such as about 0.55 millimeters (about 0.0217 inches).

In one example, preform 120 may have an inner diameter ID_{Preform} that is large enough for a loose fit with pin 94, e.g., with an inner diameter ID_{Preform} that is at least about 0.01 millimeters (about 0.00039 inches) larger than the outer diameter of pin 94, for example at least about 0.025 millimeters (0.00098 inches) larger, such as at least about 0.05 millimeters (0.00197 inches) larger than the outer diameter of pin 94. In one example, preform 120 may have an outer diameter OD_{Preform} that provides for an interference fit with pocket 104, e.g., with an outer diameter OD_{Preform} that is less than about 0.01 millimeters (about 0.00039 inches) smaller than the inner diameter ID_{Pocket} of pocket 104, for example less than about 0.005 millimeters (about 0.000197 inches), such as an outer diameter OD_{Preform} of preform 120 that is approximately equal to the inner diameter ID_{Pocket} of pocket 104.

In one example, preform 120 is configured to have sufficient polymer material so that, when melted, it forms a coating 102 that substantially fills pocket 104, as shown in FIGS. 5C and 6C. The amount of polymer included in preform 120 may be controlled by controlling the dimensions of preform 120. For example, the amount of polymer of the generally cylindrical preform 120 shown in FIGS. 5B and 6B may be controlled by selecting the inner diameter ID_{Preform}, outer diameter OD_{Preform}, and length L_{Preform} of preform 120. As discussed above, in one example the inner diameter ID_{Preform} and outer diameter OD_{Preform} of preform 120 may be selected to provide a slip fit between pin 94 and pocket 104. In one example, preform 120 has a length LP_{Preform} that is longer than a length L_{Pocket} of pocket 104. In one example, preform length L_{Preform} is between about 100% and about 500% of the pocket length L_{Pocket}, such as between about 150% and about 300% of the pocket length L_{Pocket}, for example about 200% of the pocket length L_{Pocket}. In one example, pocket 104 may have a length of about 7 millimeters, and preform 120 may have a length L_{Preform} of between about 7 millimeters and about 25 millimeters, such as between about 10 millimeters and about 20 millimeters, for example about 15 millimeters.

Preform 120 may be made from any material that is capable of substantially wetting and covering a portion of pin 94, a portion of ferrule 86, and a portion of insulation sleeve 96 to form coating 102. In one example, preform 120 comprises a thermoplastic polymer with a melting temperature that is lower than an annealing temperature of insulation sleeve 96 so that when preform 120 is melted to form coating, insulation sleeve 96 is not altered. In one example, insulation sleeve 96 has an annealing temperature of around 600° C. and preform 120 comprises a material with a melting temperature that is substantially less than the 600° C. annealing temperature of insulation sleeve 96, e.g., between about 300° C. and about 400° C. Preform 120 may also comprise a material that is chemically inert to the compounds within battery 76, such as the electrolytes within battery cell 80. In one example, preform 120 consists essentially of one or more materials that are chemically inert to the compounds within battery 76, such as the electrolytes within battery cell 80. In some examples, preform 120 comprises a material that has a melt flow index that is sufficient so that when melted, the resulting coating 102 substantially wets the entirety of portion 106 of pin 94, substantially wets the entirety of portion 108 of insulation sleeve 96, and substantially wets the entirety of portion 110 of ferrule 86, as well as substantially wetting the entirety of first junction 98 and second junction 110. Examples of thermoplastic polymers that may be used to make preform 120 include, but are not limited to, ethylene tetrafluoroethylene (ETFE), fluronated ethylene propylene (FEP), perfluoroalkoxy polymer (PFA), high-density polyethylene (HDPE), a polyethersulfone, polyetheretherketone (PEEK), or other engineered plastics such as acrylonitrile butadiene styrene (ABS), polycarbonates (PC), Polyamides (PA), polybutylene terephthalate (PBT), polyethylene terephthalate (PET), polyphenylene oxide (PPO), polyetherketone (PEK), polyimides, polyphenylene sulfide (PPS), and polyoxymethylene plastic (POM).

As its name suggests, preform 120 may be formed prior to being positioned around pin 94. Preform 120 may be formed by several methods. In one example, preform 120 is formed by extruding a thermoplastic polymer, such as ETFE or one of the other thermoplastic polymers described above, into the desired cross-sectional shape of preform 120. Extrusion is a process of producing thermoplastic components by melting a raw polymer material, and forcing the melted polymer material through a die having a cross section that is larger and proportional to the desired cross-sectional shape of the final component.

Other methods of forming preform 120, such as by molding the polymer into a desired preform shape, or punching out the desired preform shape, may be used. However, extruding preform 120 may have advantages over other methods of forming the preform shape. For example, as described above, in some examples, the inner diameter ID_{Preform} and outer diameter OD_{Preform} may be selected to provide a close, or slip, fit over pin 94 and within pocket 104. Extrusion techniques allow for greater dimensional control of the diameters ID_{Preform} and OD_{Preform}. For example, extrusion of ETFE has been shown to allow control down to within as little as about 0.005 millimeters (about 0.0002 inches) of a desired dimension. While molding, punching, or machining a part down to that level of precision is possible, it may be prohibitively expensive. Moreover, it is more difficult to mold, punch, or machine a part that is very small. For example, as noted above, in some examples preform 120 may have a desired inner diameter ID_{Preform} of between about 0.05 millimeters and about 0.55 millimeters and a desired outer diameter OD_{Preform} of between about 0.24 millimeters and about 1 millimeter. Molding, machining, or punching out a preform with these small dimensions may be difficult or expensive. Extruding the preform may also provide for economy of scale, because a long tube of the preform material may be extruded with the desired cross section and dimensions, such as generally cylindrical with the desired inner diameter ID_{Preform} and outer diameter OD_{Preform}, and then the long tube may be cut down into a plurality of preforms 120.

It is also believed that the process of extruding may provide a benefit at the molecular level. Thermoplastic parts that are molded, e.g., by injection molding, punched out, or machined tend to have a generally random distribution of the orientation of forming stresses. Extruded thermoplastic parts tend to have the forming stresses oriented generally parallel to the direction of extrusion, particularly if the neck down ratio of the polymer material (e.g., a measurement of the stretching of the polymer material, the ratio of the cross-sectional area of the extrusion die compared to the cross-sectional area of the preform, sometimes referred to as the draw ratio or draw down ratio) is relatively high. For example, ETFE has a neck down ratio of between about 80:1 and 100:1, such that the forming stresses are generally substantially oriented with the direction of extrusion, which corresponds, for example, to the axial direction of the example preform 120 shown in FIGS. 5B and 6B (e.g., in the direction of the preform length L_{Preform}).

It is believed that the orientation of the forming stresses along the axial direction of preform 120, in some examples, may allow the polymer material to better wet within pocket 104, particularly when the cross-sectional area of pocket 104, pin 94, and preform 120 are very small, e.g., as described above. While not being limited theory, it is theorized that as an extruded preform 120 is melted down and goes through annealing, glass transition, and then melting, that the generally oriented forming stresses may better allow the polymer material to become shorter and wider within pocket 104, which is believed to better wet and conform to portion 106 of pin 94, portion 108 of insulation sleeve 96, and portion 110 of ferrule. Thus, in some examples, it is believed, an extruded preform 120 may be better able to provide form complete wetting and coating, which may allow a coating 102 made from an extruded preform 120 to provide a conformal coating that avoids low leakage current between pin 94 and ferrule 86 or the formation of lithium balls.

FIGS. 5C and 6C show an example coating 102 that has been formed by melting preform 120. As best seen in FIG. 5C, in one example, coating 102 substantially covers a portion 106 of pin 94, a portion 108 of insulation sleeve 96, and a portion 110 of ferrule 86, as well as first junction 98 between pin 94 and insulation sleeve 96 and second junction 100 between insulation sleeve 96 and ferrule 86. In one example, at least a portion of coating 102 is disposed within pocket 104. In the example shown in FIG. 5C, coating substantially fills pocket 104. In one example, coating 110 is a conformal coating wherein the polymer material of coating 102 conforms to the geometry of portion 106 of pin 94, first junction 98, portion 108 of insulation sleeve 94, second junction 100, and portion 110 of ferrule 86 on interior side 88 of ferrule 86. In some examples, coating 102 may also provide a redundant seal, in addition to insulation sleeve 96, to prevent compounds within battery 76 from leaking out between pin 94 and ferrule 86.

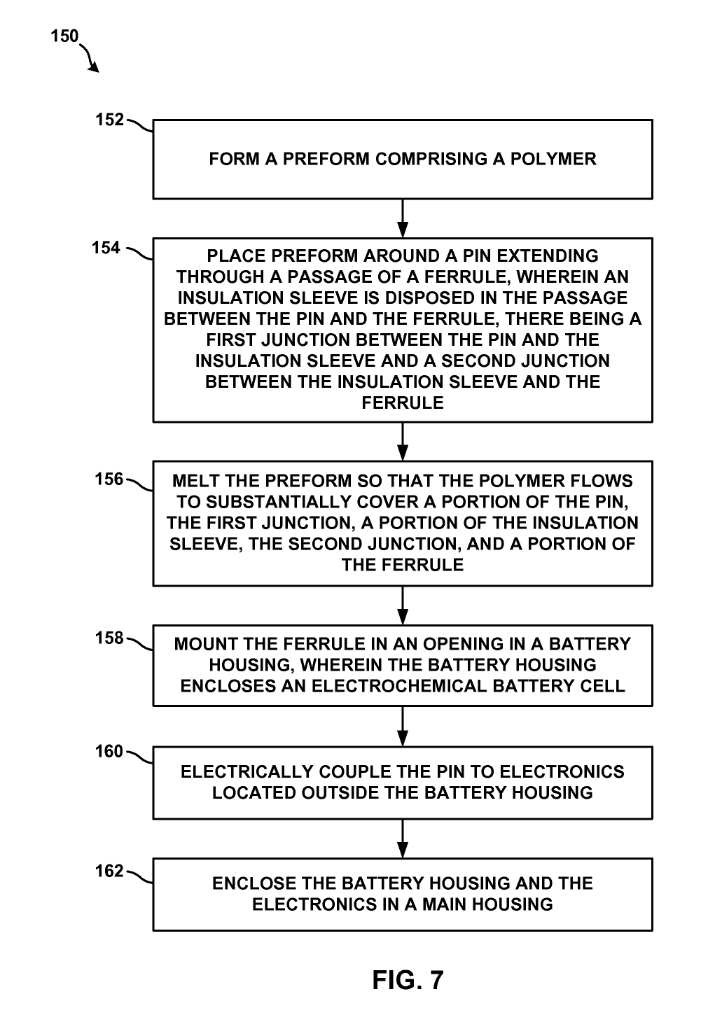

FIG. 7 is a flow diagram of an example method 150 of making a battery feedthrough 82 and IMD 70 that the feedthrough 82 may be used in. The example method 150 of FIG. 7 comprises forming a preform comprising a polymer (152), such as preform 120, placing preform 120 around pin 94 extending through a passage 92 of ferrule 86 (154), such as by inserting pin 94 through a lumen 121 of preform 120. An insulation sleeve 94 is disposed in passage 92 between pin 94 and ferrule 86 with a first junction 98 between pin 94 and insulation sleeve 96 and a second junction 100 between insulation sleeve 96 and ferrule 86. The example method 150 further comprises melting the preform so that the polymer flows to substantially cover a portion 106 of pin 94, first junction 98, a portion 108 of insulation sleeve 96, second junction 100, and a portion 110 of ferrule 86 (156). In some examples, the method 150 may also comprise mounting ferrule 86 in an opening 84 within a battery housing 80 (158), wherein battery housing 78 encloses an electrochemical battery cell 80, and electrically coupling pin 94 to electronics 74 located outside battery housing 78 (160). The example method 150 may also comprise enclosing battery housing 78 and electronics 74 in a device housing (162).

In one example method 150, forming preform 120 (152) comprises extruding the polymer to form the preform 120. As noted above, extruding the polymer to form preform 120 allows for good dimensional control over preform 120, such as over the inner diameter ID_{Preform} and outer diameter OD_{Preform} of preform 120. Extrusion also allows for the formation of a small preform 120 to be used in a small feedthrough 82, such as in a feedthrough 82 to be used in a small leadless pacemaker (e.g., IMD 16 of FIG. 1) or in a small sensor (e.g., pressure sensor 32) implantable within a heart 14, as well as the production of a plurality of preforms 120 easily and efficiently. Finally, as noted above, in some examples, extruding the polymer to form preform 120 may provide for better wetting of pin 94, insulation sleeve 96, and ferrule 86 by the polymer when melting the polymer.

In one example, feedthrough 82 comprises a pocket 104 disposed around pin 94 on a first side 88 of ferrule 86. In one example of method 150, forming preform 120 (152) comprises forming preform 120 to have a cross-sectional shape that corresponds to a cross-sectional shape of pocket 104. In one example, described above, pocket 104 has a generally cylindrical shape (e.g., a generally circular or elliptical cross section), such that forming preform (104) comprises forming a generally cylindrical preform 120 having an outer diameter OD_{Preform} that is smaller than an inner diameter ID_{Pocket} of pocket 104. In one example method 150, placing preform 120 around pin 94 (154) comprises placing at least a portion of preform 120 within pocket 104. In one example method 150, melting preform (156) comprises substantially filling pocket 104 with the polymer of preform 120 in order to substantially wet pin 94 within pocket 104, first junction 98, insulation sleeve 96 within pocket, second junction 100, and a portion of ferrule 86 within pocket 104.

In one example of method 150, melting preform 120 (156) comprises the polymer of preform 120 forming a conformal coating 102 over portion 106 of pin 94, first junction 98, portion 108 of insulation sleeve 96, second junction 100, and portion 110 of ferrule 86, e.g., wherein coating 102 substantially conforms to the geometry of portion 106 of pin 94, first junction 98, portion 108 of insulation sleeve 96, second junction 100, and portion 110 of ferrule 86. Melting preform 120 (156) may be carried out by baking preform 120, pin 94, insulation sleeve 96, and ferrule 86 in an oven in the presence of a vacuum (also referred to as vacuum baking) In one example, melting preform 120 (156) comprises baking preform 120, pin 94, insulation sleeve 96, and ferrule 86 at a temperature of between about 300° C. and about 350° C., such as between about 315° C. and about 325° C., for example about 320° C., for between about 1 hour and about 5 hours, for example between about 1 hour and about for about 3.5 hours, such as for about 70 minutes, under a vacuum.

### EXAMPLES

### Example 1

Forty eight feedthrough assemblies 82 were made, each comprising a ferrule 86 with a passage 92 therethrough, a pin 94 extending through the passage 92, and an insulation sleeve 96 disposed between the pin 94 and the ferrule 86 within the passage 92. The forty eight feedthrough assemblies 82 were placed in two fixtures, with each fixture holding twenty four feedthrough assemblies 82. Preforms made from ethylene tetrafluoroethylene (ETFE) were made by extruding ETFE (NEOFLON EP 610 grade ETFE, Daikin Industries, Ltd., Osaka, Japan) to form tubing having an inner diameter of about 0.25 millimeters and an outer diameter of about 0.55 mm. Forty eight individual preforms 120 were formed by cutting sections of the extruded tubing, with each preform 120 section having a length of about 1 mm.

Each of the forty eight preforms 120 was manually placed on a respective feedthrough assembly 82, with the pin 94 of each respective feedthrough assembly 82 being inserted through a lumen of a respective preform 120. The time to manually place the forty eight preforms 120 was measured as about 67 minutes. The two fixtures (holding the forty eight feedthrough assemblies 82 with forty eight preforms 120 placed thereon) were placed into a vacuum bake oven, where the feedthrough assemblies 82 and preforms 120 were baked for about 3.5 hours in a vacuum at a temperature of about 320° C. to melt preform 120 to form an ETFE coating 102.

The overall labor time for each feedthrough 82 with a coating 102 was about 1.4 minutes per part (about 67 minutes total for 48 parts). The overall baking time per part was about 4.4 minutes of oven time per part (about 210 minutes (3.5 hours) for 48 parts).

### Comparative Example 2

An additional forty eight (48) feedthrough assemblies were made, also comprising a ferrule 86 with a passage 92 therethrough, a pin 94 extending through the passage 92, and an insulation sleeve 96 disposed between the pin 94 and the ferrule 86 within the passage 92. The forty eight feedthrough assemblies were placed in two fixtures, with each fixture holding twenty four feedthrough assemblies. ETFE was manually coated onto the feedthrough assemblies by applying ETFE powder (TEFZEL ETFE resin powder, E.I. du Pont de Nemours and Co., Wilmington, Del.) in suspension within ethanol liquid.

In order to achieve adequate thickness and coverage of the resulting ETFE coating, a total of three coats of the ETFE powder suspension were applied to each feedthrough assembly. After each coat was applied, the feedthrough assemblies were put through a vacuum bake cycle of about 3.5 hours at a temperature of about 320° C. The application of the ETFE powder suspension required a total of about two (2) hours of labor per coat for all forty eight feedthrough assemblies, for a total of about six (6) hours of labor time for coating and a total of about 10.5 hours (3 cycles of about 3.5 hours each) of bake time.

The overall labor time to apply an ETFE coating by the method of Comparative Example 2 was about 7.5 minutes of labor per part (about 360 minutes (6 hours) total for 48 parts). The overall baking time per part was about 13.1 minutes of bake time per part (about 630 minutes (10.5 hours) total for 48 parts).

As can be seen by the comparison of the method of forming ETFE coating 102 by the method of Example 1 and the method of forming an ETFE coating by the method of Comparative Example 2, there is an average reduction in labor time of about 6.1 minutes per part and an average reduction in bake time of about 8.7 minutes per part. This translates to about an 81.3% reduction in labor and a 66.7% reduction in oven time. As an example, if the standard hourly manufacturing labor rate is about $65 per hour, then the use of the method of Example 1 will result in a labor savings of about $6.61 per feedthrough over the method of Comparative Example 2. The method of Example 1 will also result in a 66.7% reduction in the costs of operating the oven that is used for the vacuum bake.

Various examples have been described. These and other examples are within the scope of the following claims.

1. A battery feedthrough comprising:
a ferrule having a first side, a second side, and a passage extending from the first side to the second side;
a pin for conducting electricity between the first side of the ferrule and the second side of the ferrule, the pin extending through the passage;
an insulation sleeve disposed between the pin and the ferrule within the passage, wherein the insulation sleeve electrically isolates the pin from the ferrule, there being a first junction between the pin and the insulation sleeve and a second junction between the insulation sleeve and the ferrule; and
a coating comprising a polymer disposed over the pin on the first side of the ferrule, wherein the coating is formed by forming a preform comprising the polymer, placing the preform around at least a first portion of the pin on the first side of the ferrule, and melting the preform so that the polymer substantially covers a second portion of the pin, the first junction, a portion of the insulation sleeve, the second junction, and a portion of the ferrule on the first side of the ferrule.

2. The battery feedthrough of claim 1, wherein the coating conformally coats the second portion of the pin, the first junction, the portion of the insulation sleeve, the second junction, and the portion of the ferrule on the first side of the ferrule.

3. The battery feedthrough of claim 1, wherein the polymer comprises at least one of ethylene tetrafluoroethylene (ETFE), fluronated ethylene propylene (FEP), perfluoroalkoxy polymer (PFA), high-density polyethylene (HDPE), a polyethersulfone, polyetheretherketone (PEEK), an engineered plastic, acrylonitrile butadiene styrene (ABS), polycarbonates (PC), Polyamides (PA), polybutylene terephthalate (PBT), polyethylene terephthalate (PET), polyphenylene oxide (PPO), polyetherketone (PEK), polyimides, polyphenylene sulfide (PPS), and polyoxymethylene plastic (POM).

4. The battery feedthrough of claim 1, wherein the first junction provides a first hermetic seal between the pin and the insulation sleeve and wherein the second junction provides a second hermetic seal between the insulation sleeve and the ferrule.

5. The battery feedthrough of claim 1, wherein the insulator sleeve is made from an electrically insulating material comprising at least one of a boro-aluminate glass, Labor-4 glass, Cabal-12 glass, Ta-23 glass, and Corning 9013 glass.

6. The battery feedthrough of claim 1, further comprising a pocket disposed around the pin on the first side of the ferrule, wherein at least a portion of the coating is disposed within the pocket.

7. The battery feedthrough of claim 6, wherein the pocket is defined by the passage and the insulation sleeve at the first side of the ferrule.

8. The battery feedthrough of claim 6, wherein the polymer of the coating substantially fills the pocket in order to substantially cover the pin within the pocket, the first junction within the pocket, the insulation sleeve within the pocket, the second junction within the pocket, and a portion of the ferrule within the pocket.

9. The battery feedthrough of claim 6, wherein the preform has a cross-sectional shape that corresponds to a cross-sectional shape of the pocket of the ferrule so that the preform forms a slip fit within the pocket.

10. The battery feedthrough of claim 9, wherein the cross-sectional shape of the pocket is generally cylindrical having an inner diameter and wherein the cross-sectional shape of the preform is generally cylindrical having an outer diameter that is substantially the same as the inner diameter of the pocket.

11. The battery feedthrough of claim 1, wherein forming the preform comprises extruding the polymer to form the preform.

12. The battery feedthrough of claim 1, wherein the preform is generally cylindrical with a lumen extending therethrough, wherein placing the preform around the pin comprises inserting the pin through the lumen of the preform.

13. The battery feedthrough of claim 1, wherein melting the preform comprises baking the preform in a vacuum.

14. An implantable medical device comprising:
a device housing;
electronics located within the device housing, the electronics being configured to provide for a medical therapy;
a battery located within the device housing, the battery comprising a battery housing enclosing an electrochemical battery cell; and
a feedthrough comprising:
a ferrule mounted in an opening in the battery housing, the ferrule having an interior side disposed within the battery housing, an exterior side disposed outside the battery housing, and a passage extending between the interior side and the exterior side;
a pin for conducting electrical current between the electrochemical battery cell and the electronics, the pin extending through the passage;
an insulation sleeve disposed between the pin and the ferrule within the passage, wherein the insulation sleeve electrically isolates the pin from the ferrule, there being a first junction between the pin and the insulation sleeve and a second junction between the insulation sleeve and the ferrule; and
a coating comprising a polymer disposed over the pin on the interior side of the ferrule, wherein the coating is formed by forming a preform from the polymer, placing the preform around at least a first portion of the pin on the first side of the ferrule, and melting the preform so that the polymer flows to cover a second portion of the pin, the first junction, a portion of the insulation sleeve, the second junction, and a portion of the ferrule on the interior side of the ferrule.

15. The implantable medical device of claim 14, wherein the coating conformally coats the second portion of the pin, the first junction, the portion of the insulation sleeve, the second junction, and the portion of the ferrule on the first side of the ferrule.

16. The implantable medical device of claim 14, wherein the electrochemical battery cell comprises electrolytes for producing an electrical current, wherein the polymer of the coating comprises a material that is substantially chemically inert to the electrolytes.

17. The implantable medical device of claim 14, wherein the polymer of the coating comprises at least one of ethylene tetrafluoroethylene (ETFE), fluronated ethylene propylene (FEP), perfluoroalkoxy polymer (PFA), high-density polyethylene (HDPE), a polyethersulfone, polyetheretherketone (PEEK), an engineered plastic, acrylonitrile butadiene styrene (ABS), polycarbonates (PC), Polyamides (PA), polybutylene terephthalate (PBT), polyethylene terephthalate (PET), polyphenylene oxide (PPO), polyetherketone (PEK), polyimides, polyphenylene sulfide (PPS), and polyoxymethylene plastic (POM).

18. The implantable medical device of claim 14, wherein the first junction provides a first hermetic seal between the pin and the insulation sleeve and wherein the second junction provides a second hermetic seal between the insulation sleeve and the ferrule.

19. The implantable medical device of claim 14, wherein the insulator sleeve is made from an electrically insulating material comprising at least one of a boro-aluminate glass, Labor-4 glass, Cabal-12 glass, and Ta-23 glass.

20. The implantable medical device of claim 14, wherein the feedthrough further comprises a pocket disposed around the pin on the interior side of the ferrule, wherein at least a portion of the coating is disposed within the pocket.

21. The implantable medical device of claim 20, wherein the pocket is defined by the passage of the ferrule and the insulation sleeve at the interior side of the ferrule.

22. The implantable medical device of claim 20, wherein the polymer of the coating is configured to substantially fill the pocket in order to substantially cover the pin within the pocket, the first junction within the pocket, the insulation sleeve within the pocket, the second junction within the pocket, and a portion of the ferrule within the pocket.

23. The implantable medical device of claim 20, wherein the preform has a cross-sectional shape that corresponds to a cross-sectional shape of the pocket so that the preform forms a slip fit within the pocket.

24. The implantable medical device of claim 23, wherein the cross-sectional shape of the pocket is generally cylindrical having an inner diameter and wherein the cross-sectional shape of the preform is generally cylindrical having an outer diameter that is substantially the same as the inner diameter of the pocket.

25. The implantable medical device of claim 14, wherein the preform is formed by extruding the polymer to form the preform.

26. The implantable medical device of claim 14, wherein the preform is generally cylindrical with a lumen extending therethrough, wherein placing the preform around the pin comprises inserting the pin through the lumen of the preform.

27. The implantable medical device of claim 14, wherein melting the preform comprises baking the preform in a vacuum.

28. A method comprising:
forming a preform comprising a polymer;
placing the preform around at least a first portion of a pin extending through a passage of a ferrule, wherein an insulation sleeve is disposed in the passage between the pin and the ferrule, there being a first junction between the pin and the insulation sleeve and a second junction between the insulation sleeve and the ferrule; and
melting the preform so that the polymer flows to substantially cover a second portion of the pin, the first junction, a portion of the insulation sleeve, the second junction, and a portion of the ferrule.

29. The method of claim 28, wherein melting the preform comprises the polymer conformally coating the second portion of the pin, the first junction, the portion of the insulation sleeve, the second junction, and the portion of the ferrule.

30. The method of claim 28, wherein the polymer comprises at least one of ethylene tetrafluoroethylene (ETFE), fluronated ethylene propylene (FEP), perfluoroalkoxy polymer (PFA), high-density polyethylene (HDPE), a polyethersulfone, polyetheretherketone (PEEK), an engineered plastic, acrylonitrile butadiene styrene (ABS), polycarbonates (PC), Polyamides (PA), polybutylene terephthalate (PBT), polyethylene terephthalate (PET), polyphenylene oxide (PPO), polyetherketone (PEK), polyimides, polyphenylene sulfide (PPS), and polyoxymethylene plastic (POM).

31. The method of claim 28, wherein the first junction forms a first hermetic seal between the pin and the insulation sleeve and wherein the second junction forms a second hermetic seal between the insulation sleeve and the ferrule.

32. The method of claim 28, wherein the insulator sleeve is made from an electrically insulating material comprising at least one of a boro-aluminate glass, Labor-4 glass, Cabal-12 glass, and Ta-23 glass.

33. The method of claim 28, wherein there is a pocket disposed around the pin on a first side of the ferrule and wherein placing the preform around the pin comprises placing at least a portion of preform within the pocket.

34. The method of claim 33, wherein the pocket is defined by the passage of the ferrule and the insulation sleeve at the first side of the ferrule.

35. The method of claim 33, wherein melting the preform comprises substantially filling the pocket with the polymer in order to substantially cover the pin within the pocket, the first junction, the insulation sleeve within the pocket, the second junction, and a portion of the ferrule within the pocket.

36. The method of claim 33, wherein the preform has a cross-sectional shape that corresponds to a cross-sectional shape of the pocket of the ferrule so that the preform forms a slip fit within the pocket.

37. The method of claim 36, wherein the cross-sectional shape of the pocket is generally cylindrical having an inner diameter and wherein the cross-sectional shape of the preform is generally cylindrical having an outer diameter that is substantially the same as the inner diameter of the pocket.

38. The method of claim 28, wherein forming the preform comprises extruding the polymer to form the preform.

39. The method of claim 28, wherein the preform is generally cylindrical with a lumen extending therethrough, wherein placing the preform around the pin comprises inserting the pin through the lumen of the preform

40. The method of claim 28, wherein melting the preform comprises baking the preform, the pin, the insulation sleeve, and the ferrule in a vacuum.

41. The method of claim 28, further comprising:
mounting the ferrule in an opening in a battery housing, wherein the battery housing encloses an electrochemical battery cell; and
electrically coupling the pin to electronics located outside the battery housing to provide for conduction of electrical current between the electrochemical battery cell and the electronics.

42. The method of claim 41, further comprising enclosing the battery housing and the electronics in a device housing.
